(19) United States
(12) Patent Application Publication
(10) Pub. No.: US 2012/0078203 A1
(43) Pub. Date: Mar. 29, 2012

(54) Antimicrobial injection port barrier cover

(76) Inventors: Gary J. Gaube, Woodstock, CT (US); Roger E. Lapierre, Oakland, RI (US)

(21) Appl. No.: 12/930,683

(22) Filed: Jan. 13, 2011

Related U.S. Application Data
(60) Provisional application No. 61/335,871, filed on Jan. 13, 2010.

(51) Int. Cl. A61M 5/00

(52) U.S. Cl. 604/288.03; 604/288.01

(57) ABSTRACT

The invention relates to sterile medical injection catheter ports utilizing a new antimicrobial polymer mixture filled injection port barrier cover. This barrier cover, when screwed onto the injection port, protects the injection port from the colonization of microbes from the inadvertent contamination of the port with contaminated surfaces, including skin and other potential contaminants.

Cross reference to related non-provisional application No. 12/930,683. This application claims priority benefit of the provisional application No. 61/335,871 Filed Jan. 13, 2010, the entire contents of which is incorporated herein by reference.

### INVENTORS

Gary J. Gaube, 280 Child Hill Rd. Woodstock, Conn. 06281

Roger E. Lapierre, 58 Alice Ave. Oakland, R.I. 02858

### REFERENCES CITED

### U.S. Patent Documents

U.S. Pat. No. 4,343,788 Aug. 10, 1982 Mustacich

U.S. Pat. No. 6,238,575 May 29, 2001 Patil

U.S. Pat. No. 6,045,539 Apr. 4, 2000 Menyhay

U.S. Pat. No. 5,322,520 Jun. 21, 1994 Milder

### OTHER PUBLICATIONS

¹ Perencevich MD, Pittet MD. Preventing Catheter-Related Bloodstream Infections. Journal of the American Medical Assoc. 2009; 301(12):1285-1287.

² M. T. Quinn, M. S. Edwards Lifesciences F. L. Milder, Ph.D. Vantex Whitepaper 01 Science of Oligon

The invention relates to sterile medical injection catheter ports utilizing a new antimicrobial polymer mixture filled injection port barrier cover. Currently there is no catheter injection port designed for use with an antimicrobial polymer mixture barrier cover. This barrier cover when screwed onto the injection port protects the injection port from the colonization of microbes from the inadvertent contamination of the port through contact with contaminated surfaces including skin and other potential contaminants. Currently most external injection ports remain uncovered with the sealed septum membrane as the only barrier to potential contaminants while not in use. In some cases there may be a barrier cover on the injection port but this barrier cover does not contain an antimicrobial polymer mixture inside the barrier cover that is able to neutralize any potential microbes that may adhere to the injection port and begin colonization.

With the exposure of the injection port to possible contaminants the use of a time consuming bactericidal cleansing procedure is required prior to accessing the port for administration of medication. Current protocol cleaning techniques protocol involves several steps and the use of various materials for each port access. This cleaning procedure needs to be repeated each time a medication is administered through the injection port.

This is a very time consuming procedure that requires the proper cleaning materials that may not always be available.

Scientific studies have concluded that the external injection port, also referred to as the catheter hub, is the place of origin of bacterial infection. These studies only recommend that the ports are minimally handled and that the aseptic cleaning technique is properly performed each time a medication is administered utilizing the port.

All external injection ports should have an antimicrobial barrier cover over the ports to maintain the aseptic integrity of the port and medical tubing. This would reduce the risk of patient infection or other complications from subjecting the injection port to external environmental factors.

### SUMMARY OF THE INVENTION

The purpose of the invention is to provide a system that overcomes the problems of infection risk and avoids the cost and problems associated with cleaning external injection ports by providing an antimicrobial polymer mixture inside the barrier cover. The cylindrical barrier cover is open at one end and closed at the other end. It has screw threads on the inside of the cover that allows it to be screwed onto the external screw path of the catheter injection port. The utilization of a push on barrier cover is a secondary method of securing the barrier cover to the catheter injection port hub. The push on barrier cover also contains antimicrobial polymer mixture on the inside of the barrier cover. The push on cover utilizes a ribbed gripping mechanism to fasten it to the catheter injection port hub. This method is an alternate securing method to the screw on mechanism. On both methods there is a tether connecting the barrier cover to the portion of the medical venous catheter IV tubing. The antimicrobial mixture provides an aseptic barrier that neutralizes potential infectious contaminants on the injection port. The antimicrobial polymer mixture is injected or secured into the inside of the barrier cover until it fills and meets near the inner most part of the screw threads. When the antimicrobial polymer mixture is set in place it is locked into the inside of the barrier cover and forms a flexible base that acts similar to a gasket or a rubber like boot cover over the port. When the barrier cover is screwed or pushed onto the injection port hub the antimicrobial polymer mixture envelopes the port tip completely imbedding the end of the port into the antimicrobial polymer mixture. This maintains an injection port that is kept in an aseptic condition and sealed until ready for the administering of medication. This barrier cover is reusable and is replaced onto the injection port after the medication has been administered keeping the port sealed. This will maintain an aseptic port free of contamination risk and provide an immediate medication-ready port.

This simple step can be easily followed by a health care provider leaving little chance for error and assuring a safe and sterile environment for the injection port.

It is a further object of the invention to provide a protective barrier cover for an external injection port that may reduce the need for following the strict and complicated protocol of cleaning the port every time the port is accessed for the purpose of administering medication. This saves on healthcare provider time and materials.

### BRIEF DESCRIPTION OF THE DRAWINGS

FIG. 1 is an enlarged partially cut away perspective view of the external injection port barrier cover of the present invention utilizing an internal rib method of securing the antimicrobial polymer mixture to the inside of the port barrier cover.

FIG. 2 is an enlarged partially cut away perspective view of the external injection port barrier cover of the present invention utilizing an internal wedge method of securing the antimicrobial polymer mixture to the inside of the port barrier cover.

FIG. 3 is an enlarged perspective view of one type of external triple lumen catheter port for use with the present invention.

FIG. 4 is an enlarged cross-section view of an external injection barrier cover utilizing the rib method of securing the antimicrobial polymer mixture. This figure also diagrams how the antimicrobial polymer mixture forms a flexible aseptic seal over the tip of the injection port. When the barrier cover is screwed onto the port it causes the port to become imbedded into the antimicrobial polymer mixture causing the port tip to be enveloped and sealed with the antimicrobial polymer mixture.

FIG. 5 is an enlarged cross-section view of an injection port becoming imbedded into the antimicrobial barrier cover that utilizes a wedge method of securing the antimicrobial polymer mixture.

FIG. 6 is a version of FIG. 4 with the addition of a tether. The tether is to affix the barrier cover to the catheter line when the barrier cover is unscrewed and separated from the port when medications are administered.

### DETAILED DESCRIPTION OF THE DRAWINGS

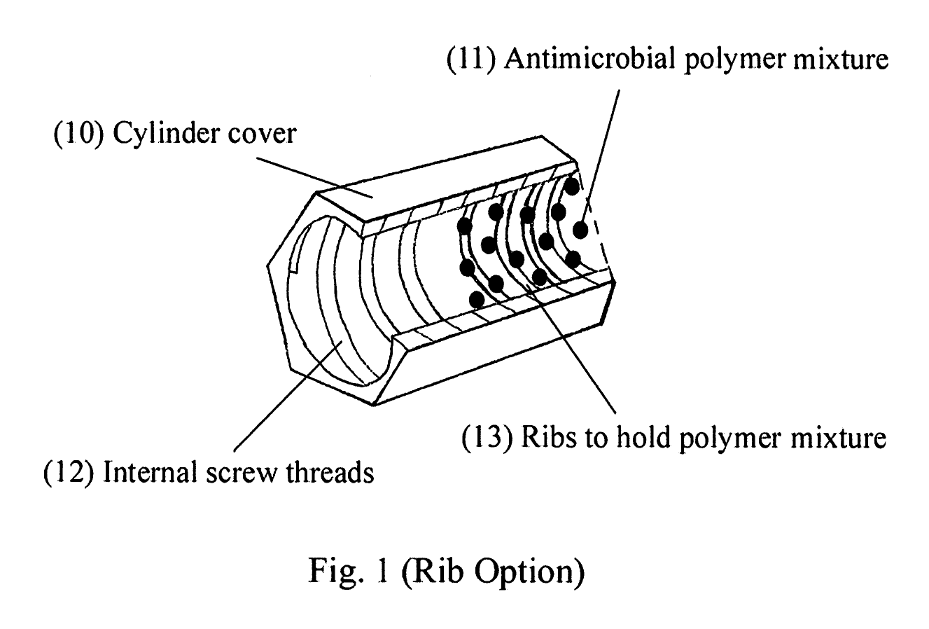

Referring to the drawing wherein like reference characters designate like or corresponding parts throughout the several views and referring particularly to FIGS. 1, 2 and 3 is seen that the invention includes a cylinder cover 10 that is open on one end and closed on the other end, having a set of screw threads 12 on the inside thereof.

FIG. 1 diagrams the ribbed method system 13 that secures the antimicrobial polymer mixture 11 to the inside of the cylinder barrier cover 10.

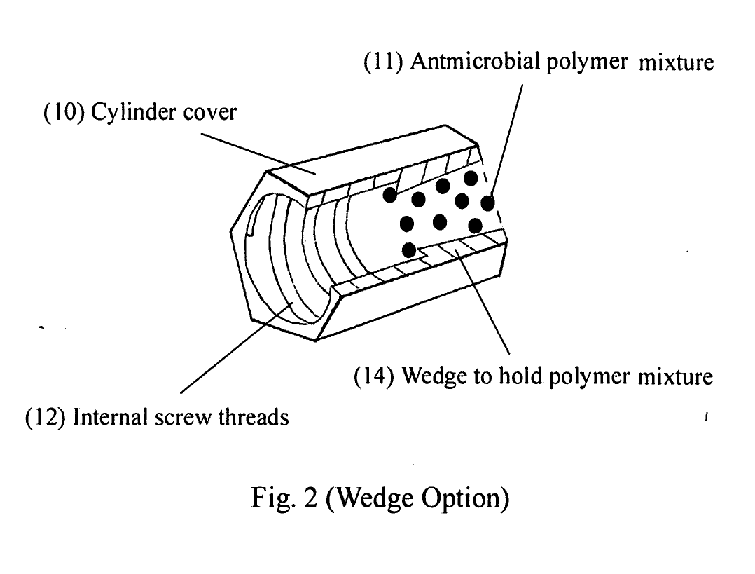

FIG. 2 is the wedge method system 14 that secures the antimicrobial polymer mixture 11 to the inside of the cylinder barrier cover 10.

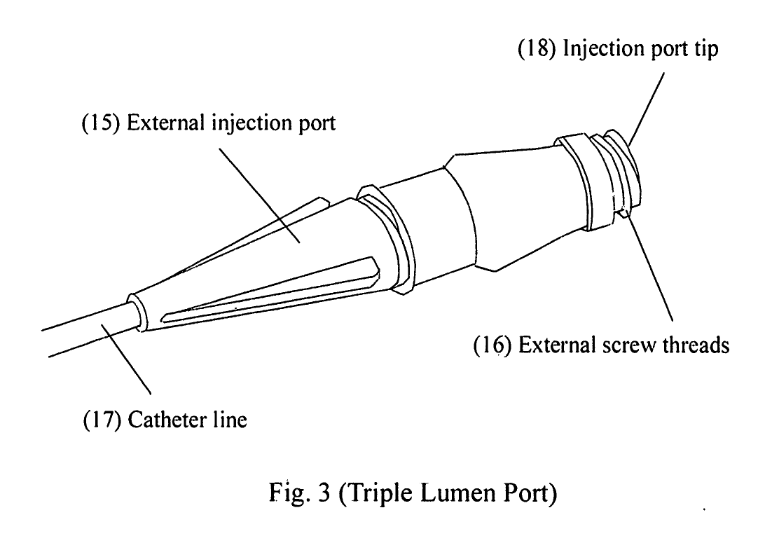

FIG. 3 diagrams the external injection port 15; it includes a set of external screw threads 16, which match up with the internal screw threads 12 on cylinder barrier cover of the invention 10 so that port 15 can be screwed into the cylinder cover 10. When said port 15 and cylinder barrier cover 10 are screwed together the injection port tip 18 becomes imbedded into the antimicrobial polymer mixture 11 creating an aseptic sealed barrier. Catheter line 17 is attached to the port 15 and leads back to the patient's catheter insertion site.

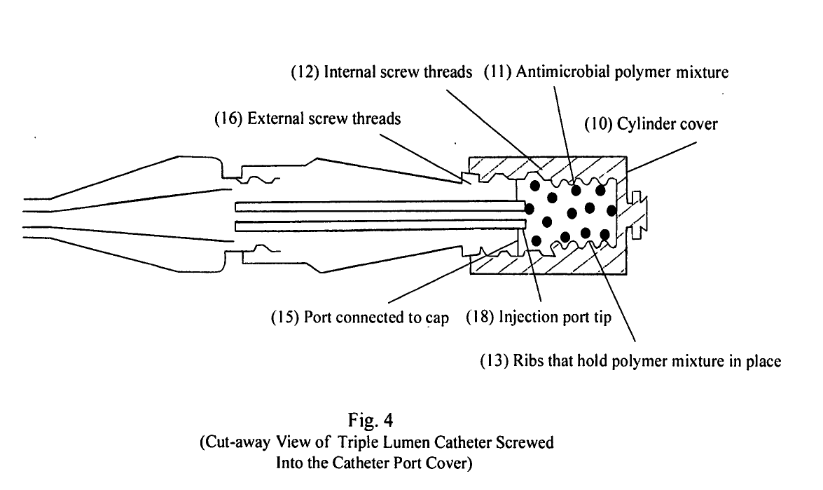

FIG. 4 is the present invention cylinder barrier cover 10 screwed onto injection port tip 18 and imbedding the port tip into the antimicrobial polymer mixture 11 creating an antimicrobial seal on the injection port tip 18. FIG. 4 utilizes the ribbed system 13 to secure the antimicrobial polymer mixture 11. The method system for securing the antimicrobial polymer mixture can be interchangeable between the rib system and the wedge system in the drawings.

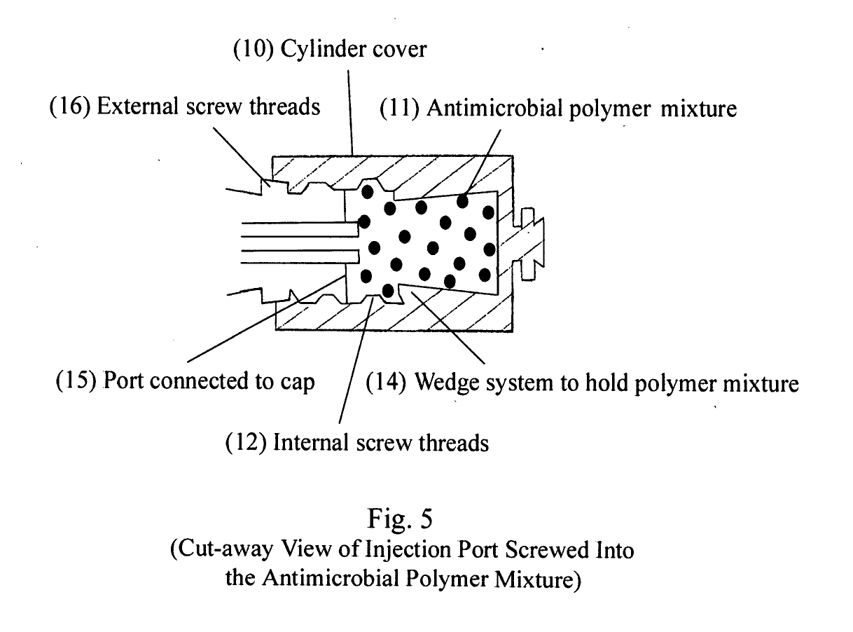

FIG. 5 is the present invention with partial view of injection port 15 showing the injection port tip 18 imbedded into the antimicrobial polymer mixture 11 that utilizes the wedge system 14 of securing the antimicrobial polymer mixture 11. This view demonstrates the injection port tip 18 being imbedded into the antimicrobial polymer mixture 11 creating an antimicrobial seal on the injection port tip 18. FIG. 5 uses the wedge system to secure the antimicrobial polymer mixture. The method system for securing the antimicrobial polymer mixture can be interchangeable between the rib system and the wedge system in the drawings.

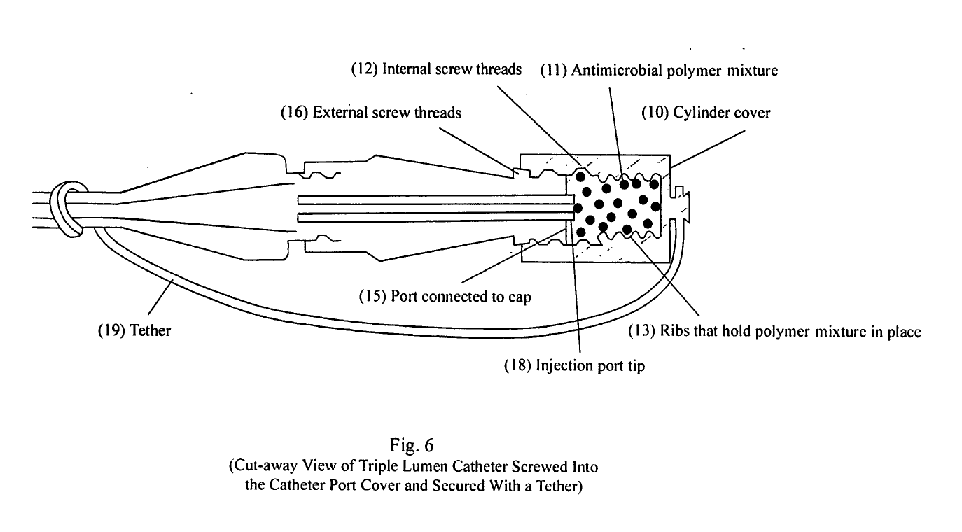

FIG. 6 is a continuation of FIG. 4 with the addition of a tether 19 to affix the barrier cover to catheter line when the barrier cover is unscrewed from the port. The tether would be included in either wedge or rib system design of securing the antimicrobial polymer mixture to the inside of the barrier cover.

### DETAILED DESCRIPTION OF THE INVENTION

In the preferred embodiment the cylindrical barrier cover of the present invention is made of lightweight rigid non-porous plastic such as polynylon. The cylinder may be of various sizes and lengths.

The helical screw threads can be of any size that is small enough to allow adequate turn circuits to assure a tight seal when the barrier cover is screwed onto the catheter port. The circumference of the inside of the barrier cover should be slightly larger than the circumference of the screw stem of the port.

As an optional fastening method of the barrier cover the utilization of a push on barrier cover is a secondary method of securing the barrier covers to the catheter injection port hub. The push on barrier cover also contains antimicrobial polymer mixture on the inside of the barrier cover. The push on cover utilizes a ribbed gripping mechanism to fasten it to the catheter injection port hub. This method is an alternate securing method to the screw on mechanism.

On both barrier cover secure methods the inner cavity of the enclosed end of the barrier cover is to contain the antimicrobial polymer mixture. This mixture may include for example polymers of latex, polypropylene or other polymer compounds.

One known antimicrobial polymer compound is Oligon.² See white paper Science of Oligon reference.

The antimicrobial Oligon relates to oligodynamic iontophoresis and more particularly to an electrically conductive structure for medical devices that reduces or eliminates bacterial infection by killing bacteria with controlled oligodynamic iontophoresis.

Oligodynamic metals, such as silver, are effective in minute quantities as bacteriostats and bacteriosiees. The most active form of these oligodynamic metals is as ions in solution. While the precise nature of the bactericidal effect is unknown, it is believed to involve altering the function of the cell membrane or linking to the cell's DNA to disrupt cell function. The bactericidal action is effective against a broad spectrum of bacteria, including all of the common strains which cause infection. When these metals are used in the minute concentrations required to kill or stem the growth of bacteria, they do not have any detrimental effect on normal mammalian cells.

Silver is used routinely in antibacterial salves, such as silver sulfadiazine, and has also been used in clinical trials to coat gauze for burn dressings. Medical devices, such as catheters, with silver impregnated in a soluble collagen or polymer coating are also known. After these catheters are placed, the coating slowly dissolves and the silver is released over time into the environment. The infection rates with these products are reported to be two to four times lower than standard catheters.

In the present invention the antimicrobial polymer mixture is to come into contact with the port hub when the barrier cap is screwed down or pushed onto the catheter port hub. The internal embodiment of the barrier cap will have a wedge system or a rib system that will secure the antimicrobial polymer insert to the inside of the barrier cap. It is also understood that the entire structure of the barrier cover could be made out of the antimicrobial polymer mixture thereby, in this instance, there would be no antimicrobial polymer insert needed. The concept of having the hub port imbedded into the antimicrobial polymer mixture when the barrier cover is fastened to the hub would remain the same.

As an example of incorporation of the antimicrobial polymer insert into the invention refer to prior art U.S. Pat. No. 5,322,520 to Milder. This prior art teaches the making of an antimicrobial polymer utilizing an Iontophoretic structure for medical devices. This iontophoretic structure for medical devices is provided that uses controlled electrical current derived from two dissimilar galvanic materials such silver and platinum to drive oligodynamic metal ions into solution to kill bacteria on and near the device to which the structure is affixed. In one embodiment, a first galvanic material separated from a second galvanic material by a resistive material produces an anti-bacterial current flow when placed in contact with an electrolytic fluid. In another embodiment, a cylindrical elastomeric catheter incorporates a first and a second galvanic material separated by a resistive material which controls a current flow between the galvanic materials when the catheter is immersed in an electrolytic fluid. The galvanic materials can be dissimilar metal powders embedded in a conductive polymer substrate that forms an iontophoretic composite material, or dissimilar metals arranged in layers separated by a resistive layer. In yet another embodiment, the iontophoretic composite material is configured as an infection control sleeve that covers a portion of an ordinary catheter or cannula. Methods of protecting implantable medical devices and body structures with the iontophoretic structures are also provided.

One additional component is needed to allow the reactions to continue and the silver ions to be released over the long term. In order that no charge build up occur on the metal powders in the polymer, an electrically conductive path between the silver and platinum particles needs to be established. This is done by also adding carbon to the polymer compound, so as to make the polymer conductive. The amount of carbon, metal powders, their ratios, their particle sizes and the permeability of the polymer composition all contribute to determining the rate of the silver ion release from the material and the longevity of the effect. For polymers used in medical devices, these parameters are adjusted to make the material a safe and effective antimicrobial for the length of the intended use of the device.

The antimicrobial polymer mixture is to be encompassed into the cavity of the enclosed end until the mixture overlaps the beginning of the screw path. The internal embodiment of the barrier cover will have a wedge system or a rib system that will secure the antimicrobial polymer mixture to the inside of the cover. In the case if the entire barrier is compounded from the antimicrobial polymer mixture then there would be no need for a compound securing mechanism.

As an example of incorporation of the antimicrobial polymer mixture into the invention refer to prior art U.S. Pat. No. 6,238,575. This prior art teaches the making of an elastic antimicrobial polymer liner for a water tank where the liner incorporates MICROBAN Additive B. with polypropylene. The polymer lining was made by first mixing pellets of MICROBAN.RTM. Additive B with polypropylene where the concentration of MICROBAN.RTM. Additive B was approximately 10% by weight. The polypropylene was polypropylene Aristech # PPT14224G manufactured by Aristech Chemical Company.

As another example of incorporating an antimicrobial polymer mixture into the invention refer to prior art U.S. Pat. No. 4,343,788 to Mustacich. This prior art teaches the making of a means for manufacturing polymers having a controlled rate of release of carboxylate antimicrobial agents of the type disclosed herein, comprising and incorporating said antimicrobial agent into said polymer and, after the polymer matrix containing the carboxylate antimicrobial is wholly (preferred) or partially cured, heating said polymer to a temperature of about 100 degrees C., or higher, in contact with moisture; for example, in a stream auto-clave, or like apparatus.

The teachings further describe several best modes in manufacturing means of the antimicrobial polymer compositions. It includes highly preferred carboxylate-permeable polymers for preparing the compositions of commercially-available silicones, especially the medical grade polydimethylsiloxanes manufactured under “clean” conditions and marketed for various medical uses. Such silicones are safe for prolonged use in contact with human tissues and provide excellent diffusion of the preferred n-octanoic and n-decanoic acid carboxylate antimicrobials.

As is well known in the art, the silicone polymers can readily be fashioned into catheters and other medical devices designed for a variety of applications. Typical examples of such silicone materials are Silastic® 382 and Dow Corning® MDA 4-4210, MDX® 4-4515, MDX® 4-4516 and Q® 7-2213, all available from the Dow Corning Corporation.

Additionally the current art teaches examples that further illustrate the preferred mode of practicing the invention using steam autoclaving and the added proton donor material to secure prolonged release of the antimicrobial agent from the polymer.

It is understood that the present invention, foregoing description and specific embodiments are merely illustrative of the best mode of the invention and the principles thereof, and that various modifications and additions may be made to the apparatus by those skilled in the art, without departing from the spirit and scope of the appended claims.

I Claim:

1-8. (canceled)

9. An antimicrobial barrier cover for an intravenous catheter hub port comprising:
a cylindrical cover comprising a first end and a second end, wherein the first end is open, and the second end is closed and may be attached to a venous catheter port by a screw thread or a push on method;
wherein said barrier cover comprises an antimicrobial soft polymer mixture in an inner portion of the barrier cover; and
wherein when said barrier cover is fastened and tightened to the catheter port the antimicrobial soft polymer mixture comes into contact with the catheter port thereby sealing the port from microbial colonization.

10. The antimicrobial barrier cover of claim 9, wherein said antimicrobial soft polymer mixture releases antimicrobial metals or metal ions, said metals or metal ions selected from the group consisting of silver, copper, zinc, tin, gold, mercury, lead, iron, cobalt, nickel, manganese, arsenic, antimony, bismuth, barium, cadmium, chromium, thallium, platinum, and combinations thereof and other antimicrobial compounds which would be compatible with the polymer mixture.

11. The antimicrobial barrier cover of claim 9, wherein said antimicrobial soft polymer mixture comprises Oligon, PolyDADMAC or other Polycationic agents that combine a broad-spectrum of antibacterial activity and may be selected from the group consisting of metal salts, antimicrobial water soluble glasses, antimicrobial metal ion-exchange type agents and combinations thereof.

12. The antimicrobial barrier cover of claim 9, wherein the antimicrobial soft polymer comprises of a variety of density, color and texture.

13. The antimicrobial barrier cover of claim 9, further comprising a solid antimicrobial polymer rod attached to the center of the inside of the cover, wherein said rod protrudes into a latex needleless port valve when fastened to a port hub to further secure a port seal.

14. The antimicrobial barrier cover of claim 9, wherein the barrier cover may be fastened to a port hub by a screw or push on mechanism.

15. The antimicrobial barrier cover of claim 9, further comprising a tether to secure the barrier cover to the body of a catheter tubing.

16. A method of incorporating an antimicrobial barrier port cover that secures to a port hub by a push on or a screw on method, wherein the entire barrier cover is manufactured utilizing a molded or extruded process that includes one or more pieces made entirely or partially from an antimicrobial polymer mixture.
